(19) United States
(12) Patent Application Publication
(10) Pub. No.: US 2022/0394978 A1
(43) Pub. Date: Dec. 15, 2022

(54) COMPOSITION AND DEVICE FOR DISINFECTING AIR

(71) Applicant: Mitchell NEWDELMAN, Monaco (MC)

(72) Inventor: Mitchell NEWDELMAN, Monaco (MC)

(21) Appl. No.: 17/836,053

(22) Filed: Jun. 9, 2022

(30) Foreign Application Priority Data
Jun. 10, 2021 (MC) 2711

(51) Int. Cl. A01N 59/00; A61L 9/014; B01J 20/20; B01J 20/26; B01J 20/10; B01J 20/28; A01N 25/12; A01P 1/00

(52) U.S. Cl. CPC A01N 59/00; A61L 9/014; B01J 20/20; B01J 20/262; B01J 20/103; B01J 20/28007; A01N 25/12; A01P 1/00; A61L 2209/22; A61L 2101/46

(57) ABSTRACT

A composition for disinfecting air including a biochar combined with a biocide. According to an embodiment, the biocide is mechanically active, i.e. it destroys, prevents the action of the pathogenic microorganism by mechanical action and without chemical action. Another aspect relates to a device for disinfecting air including the composition and a support. Another aspect relates to the use of the composition for disinfecting air or a disinfection device for reducing the amount of pathogenic microorganisms in an air flow. The present disclosure relates to the field of air disinfection. In an embodiment, it can be applied to the treatment of air for the purpose of suppressing pathogenic microorganisms such as viruses, bacteria, fungi. An embodiment can be applied in an air treatment device for treating enclosed spaces such as an entire structure or specific spaces in a building or any transport vehicle.

### TECHNICAL FIELD

The present invention relates to the field of disinfecting air. More particularly, it is applicable to the treatment of air for the purpose of suppressing pathogenic microorganisms such as viruses, bacteria and fungi. The invention can be applied to any air treatment device for treating enclosed spaces such as an entire structure or specific spaces in a building or in any transport vehicle including in air intake systems.

### PRIOR ART

The treatment of air is a long-standing problem which aims to purify air, particularly inside a building. Usually, air treatment is carried out by various known technological processes for purifying air from chemical emissions, volatile particles or even odours.

The outbreak of COVID-19 (coronavirus disease 2019), which is caused by Severe Acute Respiratory Syndrome Coronavirus 2 (SARS-CoV-2), has resulted in tens of millions of infections and hundreds of thousands of deaths. As the virus spreads in particular through the air, the treatment of air for removing airborne pathogens, in particular SARS-CoV-2 virus, has become a global health issue.

One of the solutions already known for disinfecting air includes treatments by photocatalysis. These combine a catalyst, typically titanium dioxide TiO2, with a UV-A emitter. When pathogenic microorganisms present in the air flow come into contact with the catalyst activated by UV-A radiation, the microorganisms are destroyed by OH radicals. This method works, however it is necessary that the pathogen comes into direct contact with the catalyst which limits the volumes or flow rates of air treated.

Furthermore, during this pandemic, the multiplication of disinfectant products such as hydroalcoholic gels has not made it possible to propose solutions for treating air, more precisely volumes of air since these products only allow the disinfection of surfaces. A disinfection product has recently been developed which is similar to a permanent disinfectant coating. This product is sold under the trade name Liquid Guard®. This product has a more permanent action than simple hydroalcoholic gels and other known disinfectant products. However, its only purpose is to treat pathogens that settle on surfaces and thus limit contamination through contact with skin. This product is therefore not intended for disinfecting the air.

Consequently, it is urgent to develop a solution for disinfecting air, more precisely the volume of air, which addresses the current problem of optimising air treatments.

The aim of the present invention is to provide a solution which makes it possible to disinfect the air from pathogenic microorganisms.

Other subject-matters, features and advantages of the present invention are given in the following description and accompanying drawings. Other advantages can also be included of course.

### SUMMARY

To achieve this objective, according to one embodiment the invention provides a composition for disinfecting air comprising a biochar combined with a biocide.

The combination of the biochar and biocide makes it possible to provide both a disinfectant treatment and an optimisation of the treatment surface area of the biocide due to the biochar. The invention makes it possible to treat the pathogenic microorganisms contained in a volume of air. Preferably, the disinfection of moving air.

According to particular embodiment, the biocide is configured to be mechanically active, i.e. it destroys, prevents the pathogenic microorganism from acting through its mechanical activity, preferably without chemical activity.

According to a particular embodiment, the biochar is coated or impregnated or soaked with biocide. The biochar makes it possible to considerably increase the surface area to which the biocide is applied and thus to considerably increase the available treatment surface area for a given surface.

Advantageously, the biocide applied according to the invention destroys pathogenic microorganisms by means of a mechanical process: a coating comprising nanometric points also referred to as spikes. Advantageously, positively charged nitrogen atoms attract negatively charged cell membranes. The cellular membranes of pathogenic microorganisms are then destroyed, which kills said pathogenic microorganisms preferably by perforating them. Advantageously, the spikes have a quasi-pointed, preferably pointed distal end.

Another aspect relates to a device for disinfecting air comprising the composition and a support on which the composition is deposited. The device makes it possible to reduce or suppress the number of pathogenic microorganisms contained in the air, preferably in the circulating air.

Another aspect relates to the use of the composition for disinfecting air according to the invention or a disinfection device according to the invention for reducing the amount of pathogenic microorganisms in an air flow.

Another aspect relates to a method for manufacturing the device according to the invention comprising a step of impregnating or coating the biochar with the biocide in the liquid state.

Another aspect relates to a method for disinfecting an air flow using a composition or a disinfection device according to the invention comprising the formation of an air flow by circulating the air and placing it in contact of the air flow with the biocide for reducing the quantity of pathogenic micro-organisms present in an air flow. Preferably, the biocide provides a mechanical disinfection action thanks to the kinetic action of the air flow. For example, the biocide comprises nanometric tips also referred to as spike s having a mechanical structure allowing this mechanical biocidal action by kinetic effect.

### BRIEF DESCRIPTION OF THE FIGURES

The aims, objectives, as well as the features and advantages of the invention are given in the detailed description of an embodiment thereof which is illustrated by the following accompanying drawings in which:

FIG. 1 shows the air disinfection composition according to a first embodiment: the biochar being combined with the biocide.

FIG. 2 shows a device according to an embodiment of the invention comprising the composition according to FIG. 1 in an air circulation duct.

FIG. 3 shows a device according to an embodiment of the invention comprising the composition according to FIG. 1 in an air filter.

FIG. 4 shows a device according to an embodiment of the invention comprising the composition according to FIG. 1 fixed to a support.

FIG. 5 shows a detailed view of the surface of the biocide applied to the biochar.

The drawings are given by way of example and are not limiting for the invention. They are schematic representations of principle, intended to assist with understanding the invention and are not necessarily true to scale for practical applications. In particular, the biochar and the biocide and its spikes are not representative of reality.

### DETAILED DESCRIPTION

Before giving a detailed overview of embodiments of the invention, optional features are set out below which may be used in combination or as an alternative:

According to one example, the biocide is mechanically active.

According to one example, the biochar is impregnated, soaked or coated with biocide.

According to one example, the biocide forms a solid nanometric layer, advantageously applied to the biochar.

According to one example, the solid nanometric layer has a thickness of between 10 nm and 300 nm preferably between 50 and 300 nm, preferably between 150 and 300 nm.

According to one example, the solid nanometric layer comprises spikes on its surface configured to destroy pathogenic microorganisms when the spikes come into contact with pathogenic microorganisms.

According to one example, the spikes each extend in a main direction, the cross-section of the spike, taken in a plane perpendicular to the main direction and at half the height in this main direction, having a maximum dimension Dmax of less than 25 nm (10⁻⁹ metres), and preferably less than 18 nm, Dmax being preferably between 0.01 nm and 18 nm, Dmax being preferably in the order of 0.5 nm.

According to one example, the biocide comprises a quat-silsesquioxane, also called silane quat.

According to one example, the biocide comprises silicon dioxide.

According to one example, the biocide is the product Liquid Guard®.

According to one example, the biocide is the product Zoono®.

According to one example, the biochar is hydrophilic, the biochar can have a positive, negative or neutral electrical conductivity.

According to one example, the support is an air filter provided with at least one surface through which air can flow, the composition being fixed and/or placed on at least one side of said surface.

According to one example, the support comprises an air filter defining an inner volume though which air can flow, the composition being placed in said inner volume.

According to one example, the air disinfection device is a mask for filtering air.

According to one example, the use of the device or the composition is intended for disinfecting the SARS-CoV-2 virus (Severe Acute Respiratory Syndrome Coronavirus 2).

According to one example, the method of manufacturing comprises a step of fixing the biochar on a support before or after the impregnation step.

According to one example, the method of disinfecting an air flow wherein the pathogenic micro-organisms is the SARS-CoV-2 virus (Severe Acute Respiratory Syndrome Coronavirus 2).

Biochar is a charcoal of plant origin. Biochar is a charcoal obtained by pyrolysis of the biomass of organic plant material, for example fruit or nuts shells.

The term ‘biochar’ is an abbreviation of bio-charcoar with the prefix “bio” denoting its biological origin and the English word “charcoal”.

Pyrolysis is the thermal decomposition of organic matter in an oxygen-deficient environment, leading to the production of three constituents: a gaseous mixture of non-condensable gas, bio-oil and a solid residue with a high charcoal content known as biochar. The pyrolysis preferably takes place above 180° C., for example above 200° C., preferably between 180° and 1000° C. The biochar is a charcoal which can be produced in an artisanal or industrial manner.

Biochar is known for its application as a soil conditioner.

In a conventional manner according to the International Biochar Initiative, the term biochar denotes any organic material which has been carbonised for the purpose of application onto the soil or for sequestering carbon.

Biochar is advantageously a stable carbon-rich solid which is resistant to mineralisation by microorganisms in the soil, due to its rich composition of aromatic structures. It thus acts as a carbon fixative in the soil and thus as a carbon sink, which explains why it is of interest in the context of concerns about global warming.

Biochar can be produced from organic matter of various origins (agricultural residues, manure, forestry residues, etc.).

Its use in the field of treating air contaminated with pathogens, in particular pathogens that are dangerous for human health, is not well known or even unknown.

Preferably, the biochar comprises particles of sizes between one millimetre and several centimetres in diameter. For example, from 1 mm to 15 cm. The size of the particles is defined as the largest dimension of a particle.

Advantageously, the biochar is porous. The particles of biochar comprise pores. The pores are advantageously open. The open pores are connected to the outside, either directly or via other pores and/or a network of channels or cracks. The fluids can diffuse into the open pores, which thus add to the real surface. The real surface thus corresponds advantageously to the surface in contact with the outside.

Preferably, the biochar has a porosity, more specifically pores, which have dimensions which allow the penetration of a virus. Preferably, the pores have a dimension which is greater than or equal to 18 nm, possibly 25 nm, possibly more than 100 nm.

Simply by way of example, the biochar of almond shells has a pore size range of 40 to 60 μm which is advantageous for the present invention. Thus, a contaminated air flow can enter the pores of the biochar treated with biocide according to the invention or most pathogens existing in this air flow will be captured and killed.

The biochar has an apparent surface defined by the perimeter of the particle which defines an inner volume. Advantageously, the porosity defines an inner surface, i.e. the surface of the biochar exposed to air in the inner volume. The actual surface area of the biochar exposed to air is therefore the sum of the apparent surface area and the internal surface area, advantageously accessible by nanometric pathogens contained in the air flow.

Advantageously, the porosity of the biochar is configured to define an actual surface area of in the order of 250 mm² per mm³ of biochar.

The biochar has a real surface area which is significantly greater than the visible surface area, due to its internal structure of lattices, channels and/or pores.

The dimensions of biochar particles and the porosity are configured to respect a ratio between the real surface area and the apparent surface area which is greater than or equal to 1.5, this ratio is advantageously at least 3 and preferably 10. Preferably, the ratio can be greater for large air filtration system applications.

Preferably, the biochar has a specific surface area greater than or equal to 100 m²/g and according to one possibility less than or equal to or strictly less than 500 m²/g. The specific surface area also referred to as the Mass Area corresponds to the ratio of the real surface area to the quantity of material. The mass area is determined for example by gas adsorption according to the Brunauer-Emmett-Teller or BET method.

The biochar advantageously has a carbon content greater than 70%. Usually, the charcoal has a lower carbon content. The biochar is obtained by pyrolysis with or without a subsequent activation step. The activation step can be used to suppress tars.

Preferably, the biochar is selected to be hydrophilic. This quality enables an optimised adhesion of the biocide on the biochar. Advantageously, this limits the need to use a fixing primer to improve the adhesion of the biocide.

The biochar can be exfoliated or not exfoliated. The exfoliated biochar optimises adherence to the surfaces exposed to air and more precisely exposed to a flow of air, facilitating the adherence of the biocide.

The electrical polarity of the biochar can be selected as a function of the target pathogens to optimise the capture of the pathogen. Once the polarity of a primary pathogen has been determined, the polarity of the biochar can be adjusted accordingly, to be attractive rather than repulsive. For example, according to one example embodiment, the biochar of almond shell has a low electronegative polarity.

Advantageously, the biochar is selected to have a low electronegativity to ensure a better combination with the biocide.

According to one embodiment, the biochar is hydrophobic and for example has a slightly negative polarity which retains the adhesion ability for the biocide. This also makes it possible to reduce or even avoid the risks of selection and emergence of variants surviving the biocide due to the electromagnetic force of repulsion.

According to one possibility, the composition comprises a fixing primer, forming advantageously a layer for improving the attachment of the biocide on the biochar. The fixing primer is for example an adhesion activator or promoter. For example, the fixing primer is applied in the liquid state to the biochar, once the fixing primer has dried, the biocide is applied. According to one embodiment, the fixing primer comprises for example phosphates for facilitating the adhesion of the biocide on the biochar.

The composition according to the invention also comprises a biocide. A biocide is a product which is intended to destroy, repel or render harmless, harmful organisms or prevent their action or combat them. According to the invention, a biocide acts by electrochemical or chemical or biological action or preferably mechanically.

According to a preferred embodiment, the biocide is selected to act by mechanical action, preferably only. The biocide is a mechanically acting disinfectant. Advantageously, the biocide acts by the action of kinetic energy of the pathogenic microorganism carried by an air flow which is applied to the biocide.

The disinfectant is nanometric. The disinfectant acts on the nanometric level by destroying and/or inhibiting pathogenic microorganisms.

The biocide is also understood to be an inhibitor, advantageously nanometric, preferably mechanical, of pathogenic microorganisms.

The biocide is combined with the biochar in that the biochar forms a base or support for applying the biocide. The biocide is in direct or indirect contact with the biochar, in particular via a fixing primer described below.

According to one embodiment, the biocide forms at least one coating layer of the biochar.

Advantageously, the biocide forms a solid layer with a nanometric size on the surface of the biochar, for example ranging from 150 to 300 nm. The biocide advantageously forms a layer, for example monomolecular, which binds permanently to the biochar.

The biocide is configured to infiltrate into the exposed areas of the biochar including all accessible pores, ducts and internal spaces.

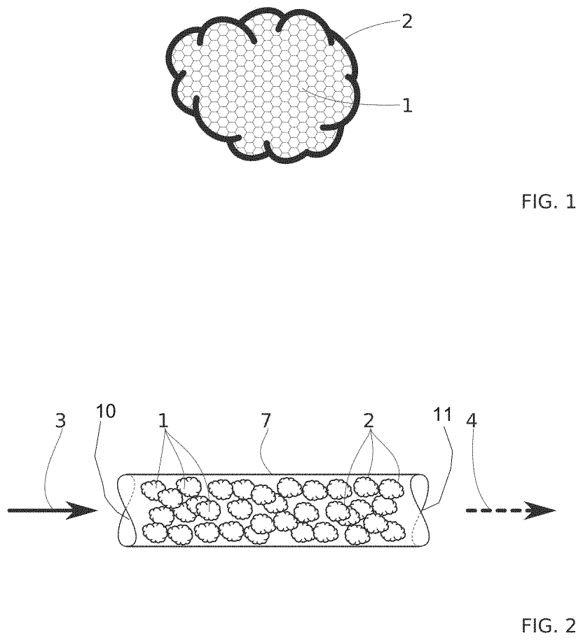

The biochar is coated or soaked or impregnated with biocide. Advantageously, the biocide coats at least the visible surface of the biochar, but also at least a part of the inner surface, i.e. the pores, particularly when the biocide has soaked or impregnated the biochar. The biocide covers at least part of the actual surface of the biochar. In this way, the biochar makes it possible to increase the application surface of the biocide and therefore optimise the treatment. Thus, the actual surface to which the biocide can be applied is much greater than the visible surface of the biochar. In FIG. 1, the biocide 2 coats the visible surface of the biochar 1 and partially the inner surface.

Advantageously, the biocide applied according to the invention destroys pathogenic microorganisms using a mechanical process: a coating comprising nanometric tips also referred to as spikes.

The biocide advantageously forms a layer comprising molecules which covalently bind to the biochar. The biocide formula layer comprising positively charged spikes. Positively charged spikes attract and pierce negatively charged microorganisms. Spikes rupture cell walls. This causes the decomposition of microorganisms with a lethal effect. Preferably, the molecules are chosen from silane-based polymers. The biocide comprising silane quats or silsesquioxane is applied to a surface. The silane-quat molecules covalently bond to the surface as the biocide dries. By using the silane base as an anchor, the long chain molecule then “stands” like a needle on the treated surface. When a pathogenic microorganism comes into contact with the bonded silane-quat molecule, the carbon-based spear consisting of the hydrocarbon methyl moiety pierces the membrane of the microorganism. Once in contact with the Silane-Quat molecule, the positively charged nitrogen molecule at the base slowly pulls the microorganism further to the hydrocarbon group and may additionally electrically neutralizes it.

According to one embodiment, the biocide comprises a nanometric structure forming spikes. Preferably, the spikes perforate the pathogenic microorganisms when they come into contact with the spikes.

According to one example the spikes each extend in a main direction. The cross-section of the spike taken in a plane perpendicular to the main direction and at half the height in this main direction, has a maximum dimension Dmax of less than 25 nm (10⁻⁹ metres), and preferably less than 18 nm, Dmax being preferably comprises between 0.01 nm and 18 nm. Dmax is preferably in the order of 0.5 nm. If the cross-section of the spike is circular, Dmax corresponds to the diameter of the spike. Preferably, the spikes have nanometric diameters for example in the order of 0.5 nm. The spikes are said to be microscopic or even nanometric. Preferably, the spike corresponds to a silsesquioxane, more precisely possibly to a terminal part corresponding to the methylated hydrocarbon chain and possibly to the central part.

According to one embodiment, the nanostructure formed in this way is composed of small points or spikes which, by a mechanical phenomenon by the energetic force of the air movement assisted or not by an electromagnetic effect, in particular by positively charged nitrogen atoms, will rise up on the arrival of a microorganism, for example naturally negatively charged, to advantageously add an electromotive force to pierce the pathogen, resulting in its instantaneous and immediate bursting on contact therewith. The disinfection is therefore mechanical and non-chemical. It is also instantaneous. The nanotechnologies can be used for forming the nanostructure, but not for forming nanoparticles from which the product is totally free.

The biocide has for example a spike density of more than 1,000 spikes/μm², preferably more than 10,0000 spikes/μm² preferably more than 500,000 spikes/μm².

According to one embodiment, the biocide comprises quat-silsesquioxane or quaternary ammonium organosilanes and/or generally known as silane quat.

The quat-silsesquioxane belongs to a group of quaternary ammoniums and is classified as a biocide in the liquid state. Its chemical name is octadecanaminium-N,N-dimethyl-N-{3-(trimethoxysilyl)propyl}-chloride. Empirical formula: C26H58ClNO3Si.

The silane quat comprises molecules comprising three parts, a base part comprises a silane for covalent bonding to surfaces, a middle part comprising a positively charged nitrogen component, and a terminal long chain part consisting of a methylated hydrocarbon group. This terminal part participates in the mechanical function of the biocide.

According to one embodiment, the biocide comprises amorphous silicon dioxide.

Preferably, the biocide is a mixture of quaternary ammonium chloride with a silane function on the alkyl group.

According to a preferred embodiment, the biocide is the product Liquid Guard® of the company Nano-Care marketed in France by the company Nano-Protection.

Liquid Guard® comprises according to one possibility quat-silsesquioxane, polymers, amorphous silicon dioxide and demineralised water.

According to one embodiment, the biocide is the product Zoono® Z-71 from Zoono. This product comprises quaternary ammoniums and more specifically organosilane quaternary ammoniums allowing disinfection by mechanical action.

The biocide is advantageously chosen to form spikes as described above which can be considered as non-flexible, semi-rigid or rigid. The biocide is advantageously chosen to form spikes as described above configured to ensure mechanical disinfection of the air flow, in particular by bursting the cell membrane of pathogenic microorganisms.

According to one aspect, the invention relates to a device for treating air comprising the composition according to the invention and a support, on which the composition is deposited.

According to one possibility, the device according to the invention comprises a fixing primer for facilitating the attachment of the biocide on the support. The fixing primer comprises possibly an adhesion activator or promoter comprising phosphates for example.

The fixing primer is advantageously placed on the support before the biocide. For example, the fixing primer is applied in a liquid state then the biocide is applied once the fixing primer has dried.

According to one aspect, the air disinfection composition and/or air disinfection device is intended for the disinfection of viruses and in particular of Severe Acute Respiratory Syndrome Coronavirus 2: SARS-CoV-2.

According to one aspect, the composition according to the invention is intended to be used with a protective mask such as a surgical mask for treating air for example.

According to one aspect, the disinfection device is an air filtration mask. The mask can be of a different type and comprises advantageously a support for the composition according to the invention, for example a filter paper or a textile or a filtration valve.

According to one aspect, the invention relates to a method for disinfecting an air flow using the composition or the air disinfection device as described in the present application. The disinfection process comprises a step of disinfecting an air flow by mechanical action. The disinfection process includes the movement of air flow, for example by a fan, by an air-conditioning system, by controlled mechanical ventilation or by natural circulation, for example by opening a window. The disinfection method further comprises the disinfection of an air flow by contact of the pathogenic microorganism(s) included in the air flow with the biocide.

According to one aspect, the invention relates to a method for preparing the composition comprising a step of impregnating or coating the biochar with the biocide advantageously in the liquid state. Preferably, the biochar is contacted with the biocide in the liquid state.

According to a first possibility, the biochar is immersed in the liquid biocide. The impregnated biochar is allowed to dry, the biocide solidifies on the visible surface and at least partially, preferably on the entire inner surface of the biochar.

Advantageously, the method uses at least 5 ml biocide per m² biochar to be coated, preferably, 10 ml, more preferably 15 ml, more preferably at least 25 ml per m².

According to one embodiment, the method for manufacturing the disinfecting device comprises fixing the composition on a support.

According to a first possibility, this fixing step is performed after obtaining the composition and therefore the step of impregnating and/or coating the biochar with biocide.

According to a second possibility, the biochar is fixed onto the support before the impregnation step and the support and biochar assembly is covered and/or impregnated with biocide.

According to one embodiment, the air disinfection device comprises the composition and a support 6. The support 6 can be varied and the composition can be fixed or not fixed to said support.

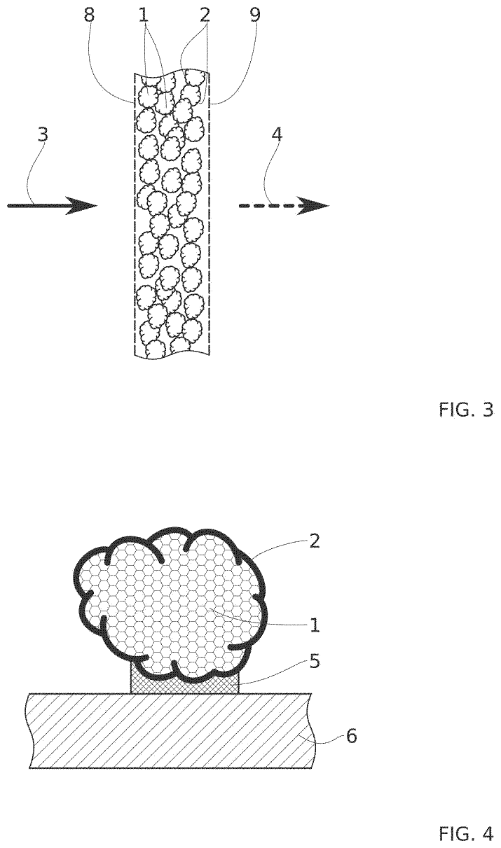

According to one possibility, the support 6 defines an inner volume in which the composition is simply placed, preferably without being fixed. The inner volume comprises advantageously an air inlet 10 and an air outlet 11. For example, as illustrated in FIG. 2, the support 6 is an air duct 7 into which the composition is simply introduced. The duct 7 can have a particular form for holding the composition in place and preventing it from being carried away with the air flow, such as for example a siphon. The airflow to be treated 3 re-enters the inner volume through an air inlet 10 and the treated airflow exits the inner volume through an air outlet 11. According to another example illustrated in FIG. 3, the support 6 comprises two filter screens 8, 9, between which the composition is embedded preferably without being fixed.

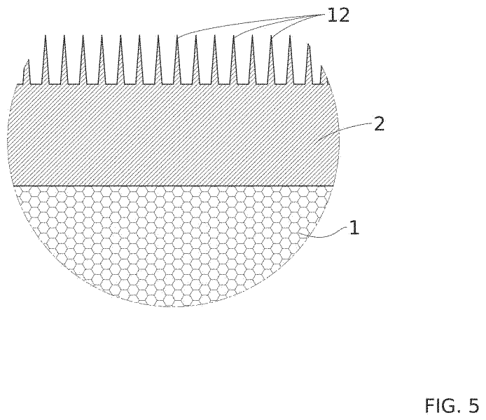

According to another possibility, illustrated in FIG. 4, the support 6 is a surface on which the composition is fixed, in particular by a fixing primer 5. The support 6 can be advantageously a filter screen.

The invention is not limited to the embodiments described above and extends to all of the embodiments covered by the invention.

### LIST OF REFERENCE NUMERALS

1. Biochar

2. Biocide

3. Incoming airflow

4. Exhaust airflow

5. Fixing primer

6. Support

7. Duct

8. Filter screen

9. Filter screen

10. Air inlet face

11. Air outlet face

12. Spike

1. A composition for disinfecting air comprising a biochar in combination with a biocide.

2. The composition for disinfecting air according to claim 1, wherein the biocide is mechanically active.

3. The composition for disinfecting air according to claim 1, wherein the biochar is impregnated, soaked or coated with biocide.

4. The composition for disinfecting air according to claim 1, wherein the biocide forms a solid nanometric layer.

5. The composition for disinfecting air according to claim 4, wherein the solid nanometric layer has a thickness between 10 and 300 nm.

6. The composition for disinfecting air according to claim 4, wherein the solid nanometric layer comprises on its surface spikes which are configured to destroy pathogenic microorganisms when the spikes come into contact with pathogenic microorganisms.

7. The composition for disinfecting air according to claim 6, wherein the spikes each extend in a main direction, the cross-section of spike, taken in a plane perpendicular to the main direction and at half the height in this main direction, having a maximum dimension Dmax lower than 25 nm (10⁻⁹ metres).

8. The composition for disinfecting air according to claim 1, wherein the biocide comprises a quat-silsesquioxane.

9. The composition for disinfecting air according to claim 1, wherein the biocide comprises silicon dioxide.

10. The composition for disinfecting air according to claim 1, wherein the biocide is the product Liquid Guard® or Zoono®.

11. The composition for disinfecting air according to claim 1, wherein the biochar is hydrophilic.

12. A device for disinfecting air comprising the composition for disinfecting air according to claim 1 and a support on which the composition is deposited.

13. The device for disinfecting air according to claim 12, wherein the support is an air filter provided with at least one surface through which air can flow, the composition being fixed and/or placed on at least one face of said surface.

14. The device for disinfecting air according to claim 12, wherein the support comprises an air filter defining an inner volume through which air can flow, the composition being placed in said inner volume.

15. The device for disinfecting air according to claim 12 being a mask intended for filtering air.

16. A method for manufacturing a device according to claim 12 comprising a step of impregnating or coating the biochar with biocide in the liquid state.

17. The method for manufacturing according to the claim 16 comprising a step of fixing biochar on a support before or after the impregnation step.

18. A method of disinfecting an air flow with a composition according to claim 1 comprising forming an air flow by circulating air and bringing the airflow into contact with the biocide for reducing the quantity of pathogenic micro-organisms present in an air flow.

19. A method of disinfecting an air flow according to claim 18 wherein the pathogenic micro-organisms is the SARS-CoV-2 virus (Severe Acute Respiratory Syndrome Coronavirus 2).

20. A disinfection device according to claim 12 comprising forming an air flow by circulating air and bringing the airflow into contact with the biocide for reducing the quantity of pathogenic micro-organisms present in an air flow.
